(19) United States
(12) Patent Application Publication
(10) Pub. No.: US 2008/0038154 A1
(43) Pub. Date: Feb. 14, 2008

(54) Method, Apparatus and Kit for Breath Diagnosis

(76) Inventors: Christopher Longbottom, Newport-on-Tay (GB); Miles John Padgett, Glasgow (GB); Kenneth David Skeldon, Glasgow (GB)

Correspondence Address: NIXON & VANDERHYE, PC, 901 NORTH GLEBE ROAD, 11TH FLOOR, ARLINGTON, VA, 22203, US

(21) Appl. No.: 10/589,872

(22) PCT Filed: Feb. 16, 2005
(86) PCT No.: PCT/GB05/00539

§ 371 Date: Jun. 29, 2007

(30) Foreign Application Priority Data
Feb. 18, 2004 (GB) 0403612.5

(51) Int. Cl. B32B 5/02; A61B 5/00; H01L 21/82

(52) U.S. Cl. 422/84; 438/132; 600/300

(57) ABSTRACT

Various medical conditions in a subject may be diagnosed by comparing the concentration of a diagnostic species such as ethane in breath samples from a subject from different stages of the breathing cycle. Using phase sensitive measurement of absorption of IR laser radiation, very small concentrations of the diagnostic species can be measured. Apparatus for collecting breath samples from different stages of the breathing cycle is also disclosed.

The present invention relates to diagnosis carried out on breath. Preferably, it relates to methods, apparatus and/or kits for carrying out breath diagnosis.

Breath analysis in medicine has been around for many years stemming from days when even the smell of a patient's breath would give doctors clues as to the patient's condition. It is known to apply different measurement techniques to analyse breath samples. Known systems for analysing breath samples make use of gas chromatography and mass spectroscopy. However, these techniques are being pushed to their useful limits. Furthermore, breath sample collection techniques are becoming more sophisticated and more time-consuming to carry out.

The hydrocarbon gas ethane is a recognised constituent in the exhaled breath of patients with a range of diseases and disorders. In particular ethane is known to be a product of excess free-radical activity associated with the development of various serious diseases and disorders. The production of ethane is related to the dynamic balance of free radical creation and scavenging in the body, termed the oxidative stress. When cells are attacked by free radicals the process of lipid peroxidation occurs and various gaseous by-products are released, volatile hydrocarbons among them.

Ethane is poorly soluble in tissue allowing its presence in exhaled breath to act as a quantitative indicator of its origin. Therefore the ability to measure the level of ethane quickly and accurately at sub part-per-billion levels gives a medically useful tool for assessing the level of oxidative stress. This is of particular interest in identifying lung cancer and cardiovascular disease.

Some of the present inventors have already developed a sensitive real-time ethane measurement system, capable of 100 parts per trillion sensitivity to ethane and a 1-second response time. Reference is made here to the paper “A field-portable, laser-diode spectrometer for the ultra-sensitive detection of hydrocarbon gases” (Gibson, G., Monk, S. D., Padgett, M., Journal of Modern Optics, 2002, Vol. 49, No. 5/6, pages 769-776), the contents of which is incorporated herein by reference in its entirety.

The present inventors have recognised that there is still a need for a technique that allows even more refined and/or accurate measurements to be carried out on breath samples. Such measurements may lead in turn to diagnoses with improved reliability. Such diagnoses should preferably be able to be performed quickly. However, some known breath diagnosis systems (e.g. gas chromatography and mass spectrometry), although relatively accurate, require significant processing of the breath sample before an analysis of the relevant composition of the breath sample can be carried out.

The present inventors have developed the present invention in order to address the drawbacks mentioned above. Preferably, the present invention reduces, ameliorates or avoids those or other drawbacks.

The present inventors have realised that a useful diagnostic indicator can be the relation between the amounts of a diagnostic species in a first breath sample compared to a second breath sample, the first sample and second sample being derived from different phases of a breathing cycle. This constitutes a general aspect of the invention.

Preferably, in a first aspect, the present invention provides a method for diagnosing a predetermined condition in a subject, said method comprising the steps of:
(i) determining the amount, or relative amount, of a predetermined diagnostic species in a first breath sample;
(ii) determining the amount, or relative amount, of said predetermined diagnostic species in a second breath sample;
(iii) relating the results of steps (i) and (ii) with the presence or absence of said predetermined condition;
   wherein said first sample and second sample are derived from different phases of a breathing cycle of said subject.

Preferably, one or more further breath samples are taken. For example, a series of n samples may be taken, where n is greater than 2. The samples may be collected, or they may be continuously sampled, e.g. in real time.

Preferably, in a second aspect, the present invention provides the use of a measurement apparatus to determine the amount, or relative amount, of a predetermined species in a first breath sample from a subject and to determine the amount, or relative amount, of said predetermined species in a second breath sample from the subject, wherein said first sample and second sample are derived from different phases of a breathing cycle of said subject.

Preferably, one or more further samples are taken, e.g. as set out with respect to the first aspect.

Preferred or optional features relating to the first and/or second aspects will now be set out. These features are independently applicable to the general, first or second aspects of the invention, and are combinable in any combination.

The subject may be human or animal, for example.

Preferably, the predetermined condition is a condition that has a discernible effect on the oxidative stress and/or lipid peroxidation in the subject. If this is the case, it is likely that the condition will thereby have an effect on the content of a measurable diagnostic species in the breath samples.

The predetermined condition may be one or more or a combination of: cancer, such as lung cancer; pulmonary disease; heart disease, cardiovascular disease or peripheral vascular disease, chronic obstructive pulmonary disease, ischaemia-reperfusion injury; Alzheimer's disease; attention deficit hyperactivity disorder; asthma; diabetes; immune and auto-immune diseases or disorders; post-organ-transplantation conditions; metabolic syndrome; stroke or other brain injury; liver disease; hyperlipidaemia; conditions resulting from hyperbaric or hyperoxia treatment; inflammatory bowel disease; vitamin E deficiency, selenium deficiency and other nutritional diseases; malnutrition; pregnancy; pre-eclampsia; gastro-intestinal conditions; or genetic disorder leading to increases in oxidative stress and/or lipid peroxidation.

Other suitable conditions will be apparent to the skilled person on reading this disclosure.

Of particular interest are lung cancer and/or cardiovascular disease.

Preferably, the first and second samples correspond to shallow (or tidal) breath and deep (or alveolar) breath. The inventors have found that gas concentration may alter over the course of the breathing cycle of a subject. Thus a sequential approach to sample collection may provide useful diagnostic indications over a measurement made on a single breath sample.

The first, second and any further breath samples may be collected as discrete samples for later analysis. Alternatively, the samples may be analysed in real time. For example, the subject's breath may be conveyed directly to a diagnostic species measurement apparatus. The first and second (and subsequent, if desired) breath samples may be considered to be samples taken in a continuous breath. Thus, for example, the determination of the amount or relative amount of diagnostic species in the subject's breath may be carried out on first, second, third, etc. samples of breath supplied to the apparatus in a continuous fashion. In preferred embodiments, this allows a graphical representation of the amount, or relative amount, of diagnostic species in the subject's breath to be plotted with respect to time (e.g. time or phase of the subject's breathing cycle).

Preferably, the diagnostic species is a species that is volatile at room temperature and pressure. Most preferably, the diagnostic species is a volatile hydrocarbon, e.g. ethane. Ethane is particularly preferred because of its low background concentration in the atmosphere.

Preferably, steps (i) and (ii) are carried out using a measurement apparatus arranged to detect the absorption of electromagnetic radiation at and/or around a known absorption wavelength for the diagnostic species. For example, the measurement apparatus may be an infra-red laser spectroscopy instrument.

Typically, the measurement apparatus has a laser source with tunable wavelength. Furthermore, the measurement apparatus may be arranged to modulate the wavelength of the electromagnetic radiation at a predetermined frequency. It may also have phase-sensitive detection means to detect intensity fluctuations of the electromagnetic radiation. Most preferably, the phase sensitive detection means is arranged to detect intensity fluctuations at an integer multiple of the predetermined frequency, e.g. two times the predetermined frequency.

Preferably, in a third aspect, the present invention provides a collection apparatus for collecting samples of breath from a subject from different phases of a breathing cycle of the subject, the apparatus having:
an inlet conduit for conveying the subject's breath;
a first collection chamber for storing a sample of breath from a first phase of the breathing cycle; and
a second collection chamber for storing a sample of breath from a second phase of the breathing cycle,
   wherein first sealable means is operable to provide a flow path from the inlet conduit to the first collection chamber and subsequently to seal the first collection chamber and second sealable means is operable to provide a flow path from the inlet conduit to the second collection chamber and subsequently to seal the second collection chamber.

The apparatus may include one or more further collection chambers for collecting samples of breath from other stages of the breathing cycle.

Preferably, in a fourth aspect, the present invention provides the use of a collection apparatus according to the third aspect to collect at least two breath samples from a subject, each sample corresponding to a different phase of a breathing cycle, including the steps:
conveying the subject's breath along the inlet conduit and into the first collection chamber via the first sealable means;
sealing the first sealable means;
conveying the subject's breath along the inlet conduit and into the second collection chamber via the second sealable means; and
sealing the second sealable means.

In the case where the collection apparatus includes more than two collection chambers (e.g. three, four or more collection chambers), the additional collection chambers preferably include associated sealable means.

Preferably, in a fifth aspect, the present invention provides a breath test kit for collecting and assessing samples of breath derived from different phases of a breathing cycle of a subject including a collection apparatus according to the third aspect and a measurement apparatus arranged to determine the amount, or relative amount, of a predetermined species in a first breath sample from the subject and to determine the amount, or relative amount, of said predetermined species in a second breath sample from the subject.

Preferred features set out with respect to the general, first and/or second aspects are independently applicable to the third, fourth and/or fifth aspect. Preferred features are set out below with respect to the third, fourth and/or fifth aspect. These are also independently applicable to the first and/or second aspect.

Preferably, the first and second sealable means allow flow of breath sample substantially in one direction only. In this way, the breath from the subject can be compartmentalised and stored in the first collection chamber and the second collection chamber without flowing back through the first and/or second sealable means.

The first and second collection chambers may have flexible walls, allowing variation of the internal volume of the chambers so that the chambers can be flattened when not in use. This allows the useful feature that, before use, the chambers contain only a small volume of gas (small in comparison to the volume of breath to be collected in each chamber) so that, after use, the chambers contain mostly breath sample rather than mostly residual gas already contained in the chambers.

Preferably, the inlet conduit and the first and second sealable means are formed of flexible materials allowing them to be flattened when not in use. This allows the collection apparatus to be stored in a small volume before use.

Typically, a mouthpiece is provided for connection to the inlet conduit. The mouthpiece may be a sterilised mouthpiece intended for a single use and then disposal. In this way, the remainder of the collection apparatus may be re-used for a different subject, e.g. using a new mouthpiece.

Preferably, the first sealable means is self-sealing and seals when a predetermined pressure is reached in the first collection chamber, subsequent breath sample thereby flowing into the second collection chamber. In this way, the volume of breath collected by the first chamber can be controlled, subsequent breath being collected in the second chamber, and/or further chambers.

An intermediate conduit may be provided between the first collection chamber and the second collection chamber. The first sealable means may be located along the intermediate conduit. Thus, when the first collection chamber is filled to the required extent, the sealing of the first sealing means may cause subsequent breath to be collected in the second collection chamber, typically upstream of the first collection chamber.

Preferably, the first collection chamber is inflated to filled volume at lower pressures than the second collection chamber. Typically, the second collection chamber is inflated by subsequent breath at a higher pressure. Most preferably, that higher pressure is insufficient to inflate the first collection chamber significantly further, but is sufficient to inflate the second collection chamber to a filled volume. For example, the second collection chamber may be formed of an elastic material. The first collection chamber may be formed of a relatively inelastic material.

In one particular embodiment, a series of collection chambers is provided, having increasing impedance routes for the gas flow. In this way, the chambers automatically inflate in a given order. For example, two, three or four (or more) collection chambers may be provided.

Additionally or alternatively, the inlet conduit may have two branch conduits, the first branch conduit leading to the first collection chamber via the first sealing means and the second branch conduit leading to the second collection chamber via the second sealing means. Typically, the second sealing means is caused to open when the first sealing means seals, allowing subsequent breath to be collected in the second collection chamber.

Preferably, the apparatus is arranged so that breath from an early phase of the breathing cycle is collected in the first collection chamber before sealing of the first sealable means and then breath from a subsequent phase of the breathing cycle is collected in the second collection chamber.

In the case where the apparatus has one or more further collection chambers, breath from one or more subsequent phases of the breathing cycle may be collected in said one or more further collection chambers.

Preferably, the apparatus includes a further collection chamber for collecting a sample of ambient air. Typically, this sample is taken at substantially the same time as the breath sample. Such an environmental sample may be useful in determining the background amount of the diagnostic species.

Additionally or alternatively, the branch conduits of the apparatus may present different flow impedances to gas flow along them. For example, they may be of different diameter. In this way, the collection bag connected to the lowest-impedance branch conduit may fill first, followed by the other collection bag(s), in order of increasing flow impedance of the respective branch conduits.

Preferred embodiments of the invention will now be described, by way of example, with reference to the drawings, in which:

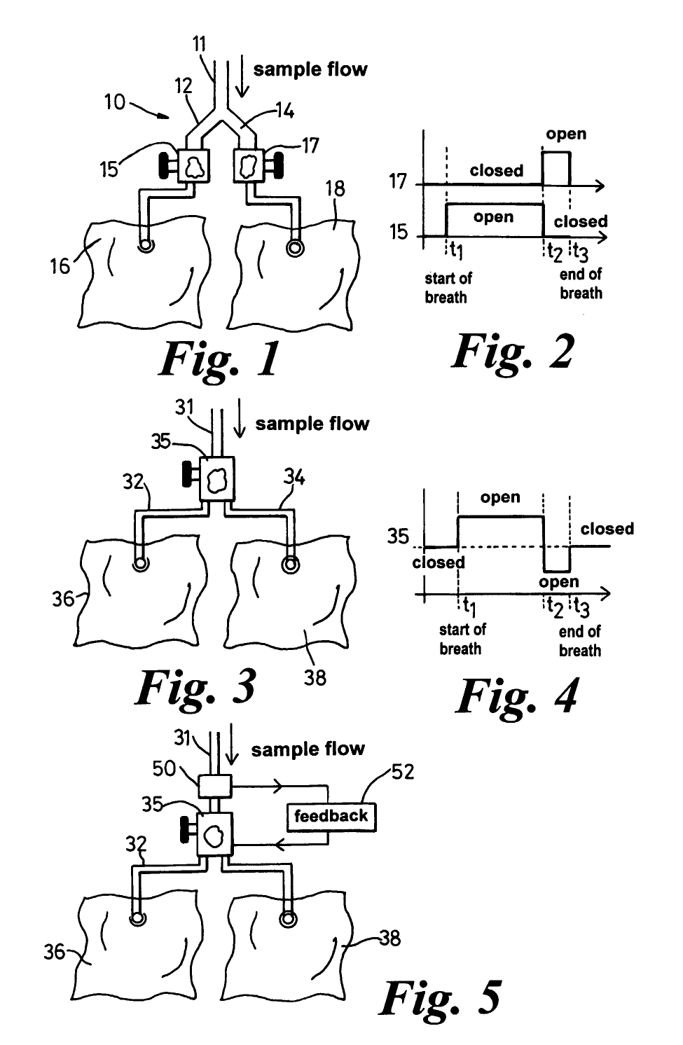

FIG. 1 shows a schematic view of an embodiment of a breath collection apparatus according to an embodiment of the invention.

FIG. 2 shows a schematic graph of the valve operation in the breath collection apparatus of FIG. 1 over a breathing cycle.

FIG. 3 shows a schematic view of another embodiment of a breath collection apparatus according to an embodiment of the invention.

FIG. 4 shows a schematic graph of the valve operation in the breath collection apparatus of FIG. 3 over a breathing cycle.

FIG. 5 shows a schematic view of another embodiment of a breath collection apparatus according to an embodiment of the invention.

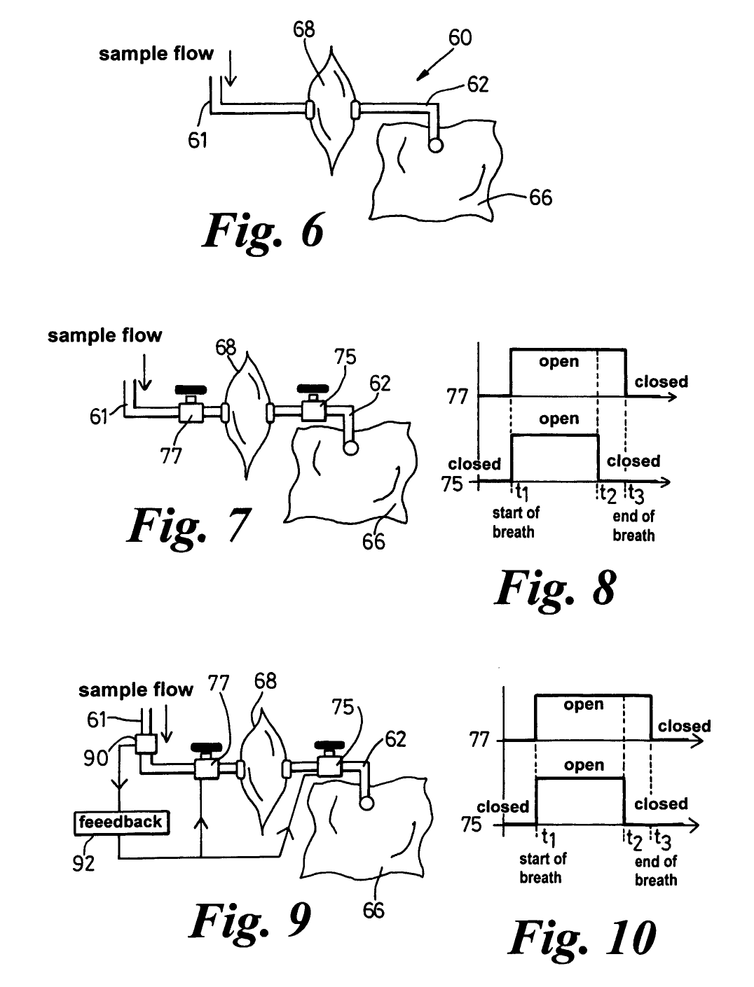

FIG. 6 shows a schematic view of another embodiment of a breath collection apparatus according to an embodiment of the invention.

FIG. 7 shows a schematic view of another embodiment of a breath collection apparatus according to an embodiment of the invention.

FIG. 8 shows a schematic graph of the valve operation in the breath collection apparatus of FIG. 7 over a breathing cycle.

FIG. 9 shows a schematic view of another embodiment of a breath collection apparatus according to an embodiment of the invention.

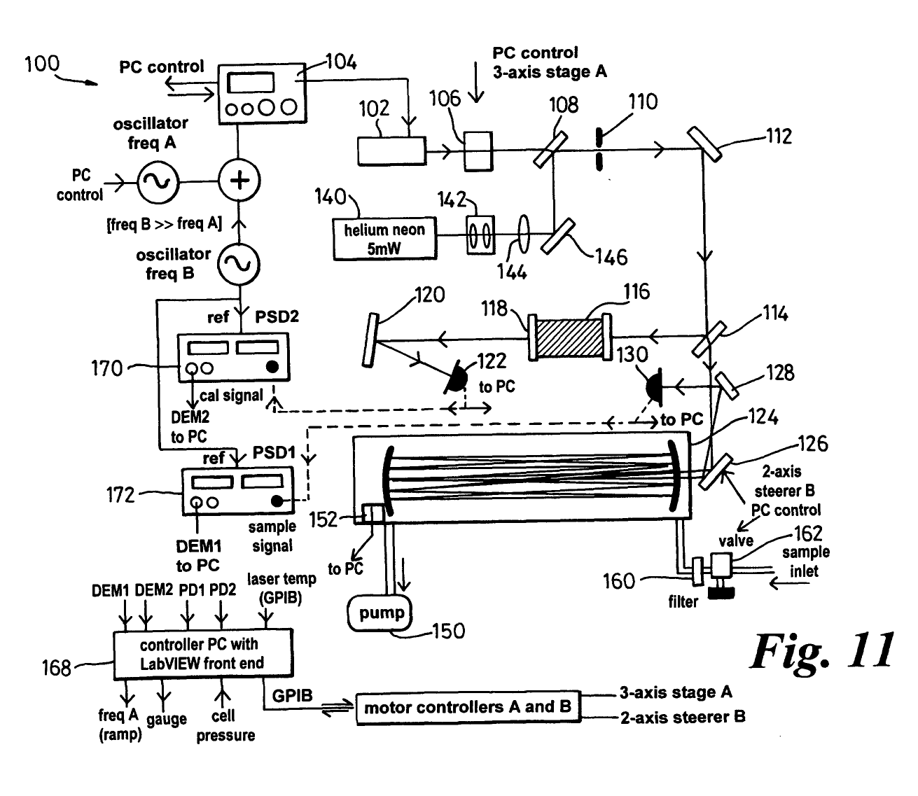

FIG. 10 shows a schematic graph of the valve operation in the breath collection apparatus of FIG. 9 over a breathing cycle.

FIG. 11 shows a schematic view of the layout of a concentration measuring instrument according to an embodiment of the invention.

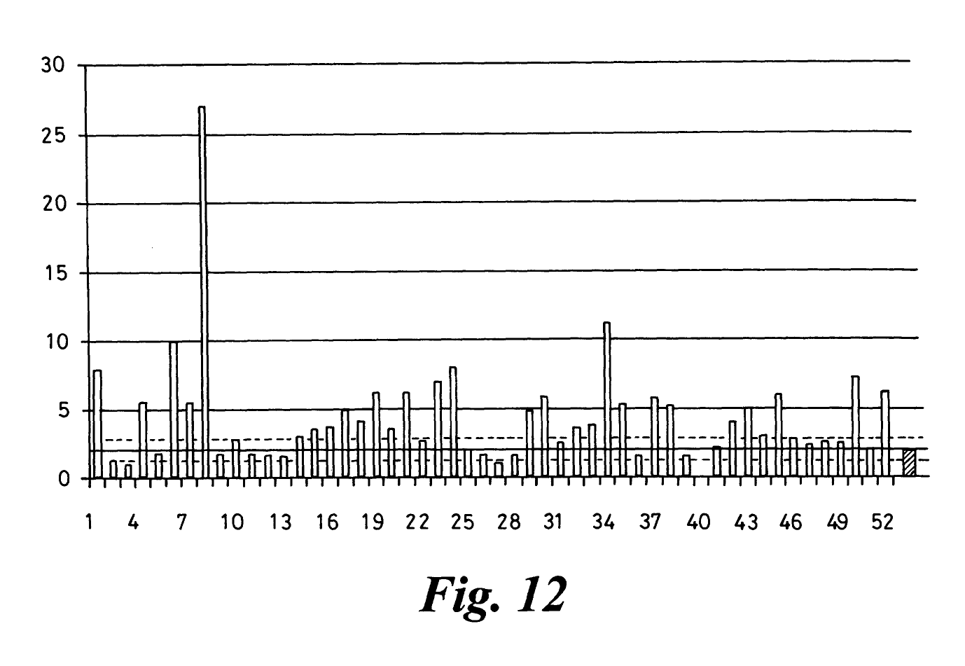

FIG. 12 shows a graph of results from a clinical trial, of use in understanding the implementation and/or application of embodiments of the invention.

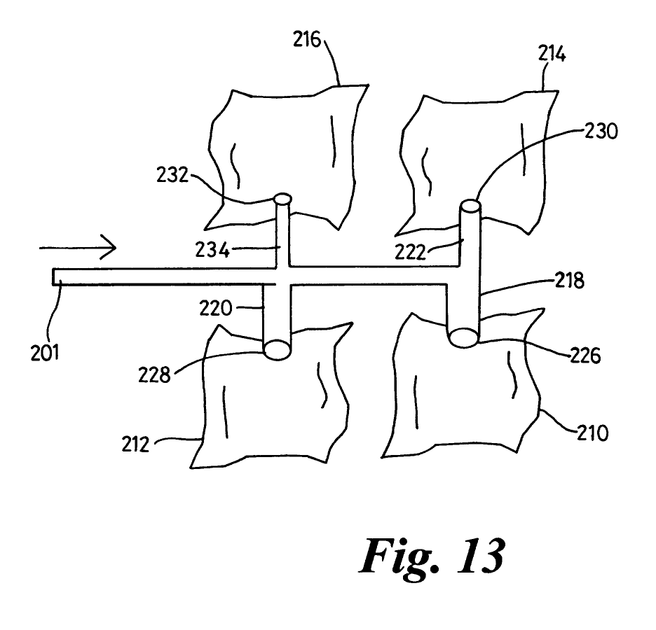

FIG. 13 shows a schematic view of another breath collection apparatus according to an embodiment of the invention.

Preferred embodiments for breath collection apparatus and uses thereof will be described first. Then, preferred embodiments for measurement of the breath samples will be described. In these preferred embodiments, the diagnostic species of interest is ethane but the present invention is not necessarily limited to measurement of ethane.

The present inventors realised that it is preferred to make the collection of the breath straightforward and non-invasive. This is in view of the practicalities accompanying screening programmes or large sample base clinical trials.

Utilising embodiments of the present invention, the ethane content of the tidal (shallow) and alveolar (deep) breath can be measured and compared. These two breath types can be collected using a variety of sample bag combinations and approaches.

Typical single breath samples are around 5 litres. Thus, for two or more breath samples taken from different phases of the breathing cycle, the cumulative volume sampled is typically around 5 litres.

Looking first at FIG. 1, there is shown a breath collection apparatus 10 having an inlet conduit 11 in the form of a tube that branches into first branch 12 and second branch 14. First branch 12 leads into first flexible collection bag 16 via first valve 15. Second branch 14 leads into second flexible collection bag 18 via second valve 17.

In use, a mouthpiece (not shown) is connected to inlet conduit 11 for a subject to breathe into. As shown in FIG. 2, at the start of the breathing cycle at time t₁, valve 15 is opened. At this time valve 17 is closed. Valve 15 is closed at time t₂. Consequently, the subject's breath between times t₁ and t₂ is collected in collection bag 16. At time t₂, valve 17 is opened. Valve 17 is then closed at time t₃, at the end of the breathing cycle of the subject. Consequently, the subject's breath between times t₂ and t₃ is collected in collection bag 18.

The timing of the opening and closing of valves 15 and 17 is set so that the subject's shallow (or tidal) breath is collected in collection bag 16 and the subject's deep (or alveolar) breath is collected in collection bag 18.

Valves 15 and 17 may be identical. Any suitable valve may be used. As will be clear to the skilled person on reading this disclosure, the pressures involved are not very much higher or lower than atmospheric pressure, so the valves may even be hand-operated tap-like valves. Alternatively, solenoid-operated valves may be used. The advantage of using solenoid-operated valves is that they may be opened and closed quickly and precisely and, optionally, automatically. For example, valves 15, 17 may be operated by a timing device, starting at time t₁ and operating as required at times t₂ and t₃.

In a preferred embodiment, bag 16 may be removable from branch 12. For example, a self-sealing valve (not shown) may connect bag 16 to branch 12. When branch 12 is disconnected from bag 16, the self-sealing valve operates to seal the bag and retain the collected breath sample therein. In this way, the collected breath sample may be stored until it can be tested, as will be described in more detail below. A similar connection may be made between bag 18 and branch 14.

FIG. 3 shows a modification of the embodiment of FIG. 1. In this embodiment, inlet conduit 31 connects directly with valve 35. Valve 35 has two outlet ports, the first connected to first branch 32 and the second connected to second branch 34. First branch leads to first flexible connection bag 36 and the second branch leads to second collection bag 38 in a way similar to the structure of the embodiment described with respect to FIG. 1.

Valve 35 has three distinct operating states. In a closed state, the valve is closed and so no breath may pass from the inlet conduit 31 to either the first branch 32 or the second branch 34. In a first open state, the valve is open to the first branch, so that breath may pass from the inlet conduit to the first branch, but not to the second branch. In a second open state, the valve is open to the second branch, so that breath may pass from the inlet conduit to the second branch but not to the first branch.

The operation of valve 35 in use is illustrated by the graph of FIG. 4. Before time t₁, valve 35 is in the closed state. At time t₁, the subject starts breathing into a mouthpiece (not shown) attached to the inlet conduit. At that time, valve 35 opens into the first open state and so breath is collected in the first collection bag 36. At time t₂, the valve switches into the second open state so that subsequent breath from the subject is collected in the second collection bag 38. Then, at the end of the breathing cycle, at time t₃, the valve is returned to the closed state.

Again, as mentioned with respect to the embodiment described with respect to FIG. 1, the valve may be hand-operated or solenoid-driven.

The embodiment illustrated by FIG. 5 is similar to the embodiment described with respect to FIG. 3. For this reason, similar features are given the same reference numbers and are not described again in detail. The modification introduced in FIG. 5 is a feedback control to determine the times t₁, t₂, t₃ at which valve 35 should operate. A flow sensor 50 is located along the inlet conduit 31, upstream of the valve 35. The flow sensor senses the flow of gas (in this case, breath) along the inlet conduit. In this way, the flow sensor is able to determine the time t₁ at which the valve 35 should be placed into the first open state. The apparatus is also capable of determining the volume of breath that has flowed passed the flow sensor, by a simple integration of the flow rates measured by the flow sensor over time. In this way, the time t₂ at which the valve should be switched into the second open state can be determined, this time being the time at which the required volume for first collection bag 36 has been collected. Furthermore, the apparatus is capable of determining when the breathing cycle has finished, because the flow rate at the flow sensor will become reduced, or will stop. This is time t₃.

The apparatus of FIG. 5 also includes a feedback loop 52 allowing control of the valve 35 based on the indications of times t₁, t₂, t₃ provided by the flow sensor 35. In this case, the valve 35 is operated via solenoid, so that opening and closing of the valve can be performed automatically, under the guidance of the feedback loop 52.

The apparatus described above can be described as having parallel collection bags. In the embodiments described below, the collection bags can be described as being in series. For the parallel collection bags, the bag material may be Tevlar. Similarly, the first collection bags of the series apparatus described below may have Tevlar bag material.

FIG. 6 illustrates another embodiment of a breath collection apparatus 60. In FIG. 6, inlet conduit 61 connects to second collection bag 68. An intermediate conduit 62 connects first collection bag 66 to the second collection bag 68. Although not shown, sealing means are provided at the connection between inlet conduit 61 and second collection bag 68 and between intermediate conduit 62 and second collection bag 68 so that, when these conduits are disconnected from the second collection bag, the sealing means operates to seal the contents of the bag. Similarly, sealing means are provided between the intermediate conduit and first collection bag 66 so that, when the intermediate conduit is disconnected from the first collection bag, the contents of that bag are sealed by operation of the sealing means.

In FIG. 6, second collection bag 68 and first collection bag 66 are not identical. Typically, first collection bag 66 is relatively easily filled by breath sample, but is also relatively inelastic so that, when filled, it will not inflate significantly further. A suitable material for the first collection bag is polyethylene. From the flattened configuration, it inflates to its inflated volume. Further inflation is not possible without a significant pressure increase, and a suitable pressure increase is normally not available when a subject is filling the bag with breath via lung-power alone. In contrast, second collection bag 68 is relatively elastic. It starts to inflate at a higher pressure than the pressure at which the first collection bag starts to inflate, but will inflate at the pressures provided by the subject when the first collection bag is filled. A suitable material for the second collection bag is an elastomer, such as rubber. In effect, the second collection bag operates as a balloon or bladder to the fixed capacity of the first collection bag.

As will be clear from the above description, when a subject breathes into the mouthpiece (not shown) attached to inlet conduit 61, the tidal breath passes through second collection bag 68 and starts to inflate first collection bag 66. At some point, the inflation capacity of the first collection bag is reached. Further inflation of the first collection bag is not possible without a significant increase in inflation pressure. However, a slight increase in the pressure provided by the subject causes the second collection bag to start to inflate, against the elasticity of the walls of the second collection bag. At this stage, the breath being provided by the subject is alveolar, so the breath collected in the second collection bag is alveolar breath.

FIG. 7 shows a modification of the embodiment described with respect to FIG. 6. Similar features are given the same reference numbers as used in FIG. 6 and are not described again in detail here.

In FIG. 7, valve 75 is provided along inlet conduit 62, between the second collection bag and the first collection bag. Valve 77 is provided along the inlet conduit, upstream of the second collection bag. Valves 75, 77 may be similar to any valve already described with respect to the embodiments described above.

The operation of valves 75, 77 is illustrated by the graph shown in FIG. 8. At the start of the breathing cycle (time t₁), valve 75 and valve 77 are opened to allow breath to pass from the inlet conduit, though the non-inflated elastic second collection bag to the first collection bag. At time t₂, valve 75 is closed to seal first collection bag 66. Subsequent breath inflates the second collection bag. At the end of the breathing cycle, at time t₃, valve 77 is closed, sealing the second collection bag 68.

FIG. 9 shows a modification of the embodiment described with respect to FIG. 7. Similar features are given the same reference numbers as used in FIG. 6 and FIG. 7 and are not described again in detail here.

FIG. 9 shows a breath collection apparatus similar to that shown in FIG. 7 except with the addition of a flow sensor 90 and a feedback control loop 92 to determine the time t₁, t₂, t₃ and to control valves 75, 77 accordingly. The use of a flow sensor and feedback control loop has been described already with respect to the embodiment of FIG. 5, so is not described further here. The operation of valves 75, 77 is illustrated by the graph of FIG. 10.

FIG. 13 shows another embodiment of a breath collection apparatus. In this embodiment, sample breath is conveyed along inlet conduit 201. Collection bags 210, 212, 214 and 216 are provided from branch conduits 218, 220, 222 and 224, respectively. Sealing means 226, 228, 230, 232 are provided between the branch conduits and the respective collection bags, so that when each collection bag is removed from its branch conduit, the sealing means is operable to seal the collection bag and retain the breath sample therein.

As is shown schematically in FIG. 13, each branch conduit 218, 220, 222 and 224 presents a different flow impedance to gas flowing along inlet conduit 201. Branch conduit 218 presents the lowest impedance (when all the collection bags are unfilled) to the flow of gas, and so collection bag 210 fills first when a subject breathes along inlet conduit 201. Once bag 210 is full, no further sample can be collected in it, so bag 212 starts filling, due to branch conduit 220 having the next-lowest impedance to gas flow. Similarly, bag 214 fills next, when bag 212 is full. Bag 216 fills last, due to the relatively high flow impedance presented by branch conduit 224.

As will be clear to the skilled person on reading this disclosure, flow sensors or gas sensors may be placed at different points along the sample flow path in order to optimise the desired filling of the sample collection bags. The sensor settings can be normalised for a given subject. This may be desirable due to the wide range of lung function of a typical range of subjects.

For analysis the two bags can be connected separately to a gas measuring instrument or the same valve system can be employed to flow breath samples into the instrument at appropriate times. Interpretation of the combined breath sample typically relies on the gas concentrations in each bag. For example it is possible to calculate the, sum, difference or ratios of the gas concentration in the two bags. The structure and operation of the gas measuring instrument is described in more detail below.

A suitable instrument for measuring the concentration of ethane in breath samples is described in detail in the paper “A field-portable, laser-diode spectrometer for the ultra-sensitive detection of hydrocarbon gases” (Gibson, G., Monk, S. D., Padgett, M., Journal of Modern optics, 2002, Vol. 49, No. 5/6, pages 769-776). This is a modified version of the Tunable Diode Laser Trace Gas Detector, an instrument available from Aerodyne Research, Inc., 45 Manning Road, Billerica, Mass. 01821-3976, U.S.A.

The standard configuration of this instrument is to use rapid scanning of the laser diode over the wavelength region of interest, thereby acquiring a transmission spectrum to which a fitting technique may be applied to give the concentration of the gas of interest.

FIG. 11 shows the layout of the concentration measuring instrument 100 according to an embodiment of the invention.

In the preferred embodiment, the optical layout is similar to that of a commercial lead salt gas sensing instrument (e.g. available from Aerodyne Research Inc.). A mid infra-red lead-salt laser diode 102) mounted within a liquid nitrogen dewar, is driven by a laser controller 104. The general wavelength of the operation is selected by setting the operation temperature of the laser diode and wavelength tuning over approximately one wavenumber achieved by direct control of the laser drive current. The laser output is collected using a 15×0.4 NA reflective microscope objective 106 that focuses the beam to a relocatable alignment pinhole 110, positioned in the back focal plane. The beam is diverted by a curved mirror 112 to a beam splitter 114 that divides the beam between a reference channel and a signal channel. The reference channel has a 100 mm long ethane calibration cell 116 with CaF₂ windows 118. The reference beam is reflected from another curved mirror 120 (radius of curvature 300 mm) to photodetector 122. The signal channel is based on an astigmatic Herriott cell 124 (Herriott, D. R., and Shultz, H. J., 1965, Appl. Optics, 4, 883) with an effective path length of over 150 m. The signal beam is directed into the Herriott cell 124 using mirror 126. On exit from the Herriott cell, the signal beam is focused using a 300 mm radius of curvature mirror 128 onto photodetector 130. Both photodetectors are cooled to improve their signal to noise performance.

As can be seen from FIG. 11, a further modification is the use of an alignment laser 140. A beam splitter 108 before the pinhole 110 is used to divert part of the main beam. The beam is aligned by use of mirror 146, lens 144 and beam expander 142.

The Herriott optical delay line allows in excess of 100 transits of the signal beam. The pressure within the sample cell can be maintained at a low level (to be determined by the user) using pump 150 and pressure gauge 152. Typically, the cell pressure is maintained at 30 torr using an oil-free, scroll pump but can be altered to provide different inward flow rates to the sample cell.

The preferred embodiment uses two micro-positioning devices. The first, positioning stage A, enables the alignment of the microscope objective to be precisely controlled. The second, steering device B, is used to provide a two-axis motion for a mirror mount for controlling the position of mirror 126 which couples the light both into and out of the sample cell. These positioning devices are of a particular design whereby when powered off they remain in their existing position and possess stability comparable to standard, high-quality, mirror mounts.

The sample is drawn into the Herriott cell via an in-line PTFE dust filter 160 through toggle valve 162 from a subject directly, the environment, or a sample chamber e.g. a bag as described with respect to FIG. 1-10, or 13. Sampling may be continuous, the pump speed and system volume resulting in a time constant for the gas mix in the cell of approximately 2 seconds. The toggle valve 162 on the inlet allows the input to be remotely switched from sample intake to a clean nitrogen source enabling an accurate zero reference to be maintained.

The pressure in the sample cell is defined by a control valve on the sample intake side of the cell and the vacuum pump. The resulting flow rate at the intake port is roughly 5 litres per minute. Therefore, a gas sample bag of 5 litre volume will give one minute of useful absorption data for the instrument to analyse. In practice, the volume of the cell is about 51 also, and therefore takes about 3 to 4 seconds to completely fill with new gas. This can be regarded as the response time of the instrument, although in the steady state condition, where it is monitoring a constant amount of gas, it is the scan rate of the laser that dictates the useful measurement sample rate. This can range from below 1 Hz to a few Hz with some compromise with regard to instrument sensitivity. With these factors all considered the sample rate of the instrument can be defined as equivalent to the laser scan rate, typically 1 Hz. The response time on the other hand is a function of the pressure in the cell, and is typically 4 seconds. These parameters allow the measurement of gas samples from bags containing only around 1 litre of volume. The instrument also allows measurement of samples from subjects in real time as they blow into the instrument, because the response time is sufficient to see any ethane rise associated with the latter stage of the breathing cycle.

The instrument is controlled by a personal computer 168 using custom designed user interface based on the LabVIEW programming environment. A phase sensitive detector (PSD2) 170 measures the calibration signal from photodetector 122 of calibration cell 118. Another phase sensitive detector (PSD1) 172 measures the signal from photodetector 130 of the Herriott sample cell. Signal DEM2 from PSD2 170 and signal DEM1 from PSD1 172 are sent to PC 168. Other signal from the other addressable components of the instrument are also sent to PC 168. Furthermore, the 3-axis stage A and the 2-axis steerer B can be controlled from the PC.

The lead salt laser 104 has wavelength closely matched to several main absorption transitions for ethane gas and other hydrocarbons. The laser wavelength is ramped at around 1 Hz to a few Hz (frequency A) centred on a strong ethane absorption line. Inside the Herriott cell the light makes over 100 transits before exiting to be detected by photodetector 130. The change in light intensity due to absorption by ethane has a transfer function of about 0.025% per ppb of concentration. In order to reliably measure such a small signal change we employ a wavelength modulation measurement technique and use lock-in detection at the second harmonic. In this way, the signal is zero when there is no ethane trace and has a well-defined peaked shape as a function of the wavelength sweep when ethane is present. With this technique, and careful signal fitting and analysis a sensitivity limit of 100 parts per trillion is obtained, which is around a factor of a few higher than the theoretical shot noise limit given the typical amount of light we detect.

Turning now to the use of the instrument described above for use in measuring ethane concentration in breath samples, a clinical trial is described that will be of use in understanding the mode of operation and implementation of preferred embodiments of the present invention.

50 patients were targeted on their first referral visits to the pulmonary clinic at Ninewells Hospital in Dundee, Scotland, U.K. A single breath sample was taken from each patient along with some supplementary written data such as the time since last cigarette. For the most part, samples were taken from patients while waiting to be seen by the consultant, and before they took part in other tests. The patients were asked to blow into 51 Tedlar sample bags through disposable mouthpieces. The bags were then sealed and stored for later analysis. The time taken for each sample, including the acquisition of the supplementary data, was about 3 to 4 minutes.

A matched control set was accessed through the dental school at the University of Dundee, Scotland, U.K. This allowed, in the dental practice setting, the investigation of the acquisition of breath samples from a section of the population. Secondly, the spectrum of ethane results for these controls was expected to show whether obtaining samples in a dentistry setting would prove acceptable in view of various possible factors that could affect the ethane result. For example, consideration was required of the possibility of contaminants in the atmosphere from various chemicals used in the dental surgery that could have an impact as subjects come into respiratory equilibrium with the air. There is also the (largely unknown) contributions possibly effected by basic dental problems and general patient apprehension of attendance in the first place.

A graph of results from this clinical trial is shown in FIG. 12. The abscissa of the graph represents the identifying number of the breath test corresponding to particular individuals taking part in the trial at Ninewells Hospital. The ordinate of the graph represents the concentration of ethane in the breath samples, in parts per billion. The bar at the right-hand end of the graph represents the average concentration of ethane in the breath of the control subjects which was 2.27+/−0.9 parts per billion.

The results shown in FIG. 12 indicate that the ethane level from the patients with a range of medical disorders are clearly higher than this average value for our controls. On our trial, the patients were diagnosed as having COPD, TB, lung cancer among other conditions.

It should be noted that ethane is not generated by ecological mechanisms in the same way as, for example, methane. This results in a much lower environmental background level (about 1 ppb) for ethane. The low background levels of ethane in the atmosphere further facilitates the medical application of the present technology, because in the preferred embodiment of the use of the invention, typical raised levels of order a few ppb are being compared with the low residual level present even in the lungs of healthy individuals.

The results described above are encouraging in view of the potential to screen for serious disease in a realistic manner. They are of particular importance for screening for lung cancer. The progress of this disease is often insidious and in the majority of cases, a diagnosis comes late and usually after the cancer is no longer confined. For example, with present diagnostic figures the 5-year survival rate for newly identified cases is less than 5%. On the other hand, reports indicate that earlier detection of the condition would lead to 5-year survival rates of over 50% and a significant number beyond that.

The present technology also has applications in other areas of healthcare outside the area of screening or diagnosis. There are various areas where monitoring the oxidative stress of a patient is beneficial. The oxidative stress mechanism within the body can have a very small time constant, and so the constant monitoring of ethane could give useful indication of a patient's general state of health. This has applications in various areas, for example:—

(1) Monitoring patients' ethane levels in Intensive Care Units as a signature of general well-being.

(2) Monitoring patients' ethane levels during surgery as an indicator of well being.

(3) Implementing ethane breath tests for the effectiveness of hyperbaric oxygen treatment (a process specifically tailored to reduce free radical activity in the body).

(4) Monitoring the oxidative stress as a function of age.

(5) Testing the effectiveness of drugs designed to reduce oxidative stress levels by measuring patients ethane in the breath.

(6) Monitoring ethane levels in animals as a non-invasive research tool.

(7) Monitoring ethane levels in race horses and dogs as an indicator of respiratory condition.

(8) Monitoring ethane levels as a function of diet.

(9) Monitoring the oxidative stress in low life-span species, for example birds, as a research tool for investigating oxidative stress and related factors over a life cycle. The embodiments described above can be modified to allow measurement of a second or further diagnostic species, e.g. a gas that gives significant additional information on a subject's medical condition. For example, the amount of methane may be measured as a second diagnostic indicator.

The embodiments above are described by way of example. Modifications of these embodiments, further embodiments and modifications thereof will be apparent to the skilled person in the light of this disclosure and, as such, are within the scope of the invention.

1-22. (canceled)

23. A method for diagnosing a predetermined condition in a subject, said method comprising the steps of:
(i) determining the amount, or relative amount, of a predetermined diagnostic species in a first breath sample;
(ii) determining the amount, or relative amount, of said predetermined diagnostic species in a second breath sample;
(iii) relating the results of steps (i) and (ii) with the presence or absence of said predetermined condition;
wherein said first sample and second sample are ex vivo and are derived from different phases of a breathing cycle of said subject, the first breath sample being of tidal breath and the second breath sample being of alveolar breath, and steps (i) and (ii) are carried out using a measurement apparatus arranged to detect the absorption of electromagnetic radiation at and around a known absorption wavelength for the diagnostic species.

24. A method according to claim 23 wherein the predetermined condition is a condition that has a discernible effect on the oxidative stress and/or lipid peroxidation in the subject.

25. A method according to claim 23 wherein the predetermined condition is one or more or a combination of: cancer, such as lung cancer; pulmonary disease; heart disease, chronic obstructive pulmonary disease, cardiovascular disease or peripheral vascular disease, ischaemia-reperfusion injury; Alzheimer's disease; attention deficit hyperactivity disorder; asthma; diabetes; immune and auto-immune diseases or disorders; post-organ-transplantation conditions; metabolic syndrome; stroke or other brain injury; liver disease; hyperlipidaemia; conditions resulting from hyperbaric or hyperoxia treatment; inflammatory bowel disease; vitamin E deficiency, selenium deficiency and other nutritional diseases; malnutrition; pregnancy; pre-eclampsia; gastro-intestinal conditions; or genetic disorder leading to increases in oxidative stress and/or lipid peroxidation.

26. A method according to claim 23 wherein the diagnostic species is a species that is volatile at room temperature and pressure.

27. A method according to claim 23 wherein the measurement apparatus has a laser source with tunable wavelength.

28. A method according to claim 27 wherein the measurement apparatus has phase-sensitive detection means to detect intensity fluctuations of the electromagnetic radiation from the laser source.
